(19) United States
(12) Patent Application Publication
(10) Pub. No.: US 2025/0312360 A1
(43) Pub. Date: Oct. 9, 2025

(54) MITOCHONDRIAL ANTIOXIDANT TREATMENT FOR CASK-LINKED NEURODEVELOPMENTAL DISORDERS

(71) Applicant: Virginia Tech Intellectual Properties, Inc., Blacksburg, VA (US)

(72) Inventors: Konark MUKHERJEE, Blacksburg, VA (US); Sarika SRIVASTVA, Blacksburg, VA (US)

(21) Appl. No.: 18/865,520

(22) PCT Filed: May 12, 2023
(86) PCT No.: PCT/US2023/022032

§ 371 Date: Nov. 13, 2024

Related U.S. Application Data
(60) Provisional application No. 63/341,816, filed on May 13, 2022.

(51) Int. Cl. A61K 31/66; A61K 31/14; A61K 31/23; A61K 31/352; A61K 31/4375; A61P 25/28

(52) U.S. Cl. CPC A61K 31/66; A61K 31/14; A61K 31/23; A61K 31/352; A61K 31/4375; A61P 25/28

(57) ABSTRACT

Described in certain example embodiments herein are methods of and compositions for treating a CASK-linked neurodevelopmental disorder. In certain example embodiments. the method includes administering. to a subject in need thereof. an amount of an SkQ compound or formulation thereof.

### CROSS-REFERENCE TO RELATED APPLICATIONS

This application claims the benefit of and priority to co-pending U.S. Provisional Patent Application No. 63/341,816, filed on May 13, 2022, the contents of which is incorporated by reference herein in its entirety.

### STATEMENT REGARDING FEDERALLY SPONSORED RESEARCH

This invention was made with government support under Grant No. EY024712 and NS117698 awarded by National Institutes of Health. The government has certain rights in the invention.

### TECHNICAL FIELD

The subject matter disclosed herein is generally directed to treatments for Pontocerebellar hypoplasias (PCH).

### BACKGROUND

Pontocerebellar hypoplasias (PCH) are a group of disorders characterized by a thin cerebellum and brain stem. PCH are neurodegenerative conditions that begin prenatally and have a poor prognosis. Overall, there have been very few investigations into the mechanism of PCH, and there are no existing therapies. As such there exists an urgent need for improved understanding of PCH and PCH treatments.

Citation or identification of any document in this application is not an admission that such a document is available as prior art to the present invention.

### SUMMARY

Described in certain example embodiments herein are methods of treating a CASK-linked neurodevelopmental disorder and/or a symptom thereof in a subject in need thereof, the method comprising administering an amount of an SkQ compound or formulation thereof to the subject in need thereof.

In certain example embodiments, the SkQ compound is selected from SkQ1, SkQR1, SkQ2, SkQ2M, SkQ3, SkQ4, SkQ5, SkqBerb, SkQPalm. Ct12TPP, MitoQ, and any combination thereof.

In certain example embodiments, the CASK-linked neurodevelopmental disorder and/or a symptom thereof is a cerebellar hypoplasia.

In certain example embodiments, the CASK-linked neurodevelopmental disorder and/or a symptom thereof is a Pontocerebellar hypoplasia (PHC). In certain example embodiments, the PHC is a PHC type 1, a PHC type 2, PHC type 3, PHC type 4, PHC type 5, or PHC type 6. In certain example embodiments, the PHC type 1 is PHC type 1A or PHC type 1B. In certain example embodiments, the PHC type 2 is PHC type 2A, PHC type 2B, or PHC type 2C.

In certain example embodiments, the CASK-linked neurodevelopmental disorder and/or a symptom thereof is microcephaly with pontine and cerebellar hypoplasia.

In certain example embodiments, the CASK-linked neurodevelopmental disorder and/or a symptom thereof is an X-linked intellectual disability.

In certain example embodiments, the CASK-linked neurodevelopmental disorder and/or a symptom thereof is epilepsy.

In certain example embodiments, the CASK-linked neurodevelopmental disorder and Rett syndrome.

In certain example embodiments, administration of the SkQ compound or formulation thereof occurs during a therapeutic temporal window.

In certain example embodiments, mitochondrial metabolism is improved in the subject in need thereof as measured by a decrease in brain reactive oxygen species in the brain, an increase in brain mitochondrial basal respiration, increase in brain glucose oxidation, a decrease in brain lipid oxidation, an increase in the levels of brain arachidonic acid as compared to the subject in need thereof prior to administration or other suitable control or threshold value.

In certain example embodiments, neuron loss, neuron dysfunction, neurodegeneration, or any combination thereof is decreased in the subject in need thereof after administration as compared to the subject in need thereof prior to administration or other suitable control or threshold value.

Described in certain example embodiments herein are pharmaceutical formulations for treating a CASK-linked neurodevelopmental disorder or a symptom thereof in a subject in need thereof, the pharmaceutical formulation comprising an amount of an SkQ compound effective to treat a CASK-linked neurodevelopmental disorder or a symptom thereof; and a pharmaceutically acceptable carrier.

In certain example embodiments, the SkQ compound is selected from SkQ1, SkQR1, SkQ2, SkQ2M, SkQ3, SkQ4, SkQ5, SkqBerb, SkQPalm. Ct12TPP, MitoQ, and any combination thereof.

In certain example embodiments, the CASK-linked neurodevelopmental disorder and/or a symptom thereof is a cerebellar hypoplasia.

In certain example embodiments, the CASK-linked neurodevelopmental disorder and/or a symptom thereof is a Pontocerebellar hypoplasia (PHC).

In certain example embodiments, the PHC is a PHC type 1, a PHC type 2, PHC type 3, PHC type 4, PHC type 5, or PHC type 6.

In certain example embodiments, the PHC type 1 is PHC type 1A or PHC type 1B.

In certain example embodiments, the PHC type 2 is PHC type 2A, PHC type 2B, or PHC type 2C.

In certain example embodiments, the CASK-linked neurodevelopmental disorder and/or a symptom thereof is microcephaly with pontine and cerebellar hypoplasia.

In certain example embodiments, the CASK-linked neurodevelopmental disorder and/or a symptom thereof is an X-linked intellectual disability.

In certain example embodiments, the CASK-linked neurodevelopmental disorder and/or a symptom thereof is epilepsy.

In certain example embodiments, the CASK-linked neurodevelopmental disorder and Rett syndrome.

Described in certain example embodiments herein are kits comprising the pharmaceutical formulation of the present description herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1B—MRI scans of CASK-linked PCH. (FIG. 1A) Magnetic resonance imaging (MRI) brain scan of a female with heterozygous mutation (G659D) in CASK. (FIG. 1B) MRI brain scan of a male with hemizygous mutation in CASK R27*. Note the severity in the male.

FIG. 2A-2H—Postnatal microcephaly and cerebellar hypoplasia in CASK^{(+/-)} mice. CASK^{(+/-)} heterozygous mutant mice display postnatal microcephaly and cerebellar hypoplasia. (FIG. 2A) Representative brain images of sex-matched CASK^{(+/+)} and CASK^{(+/-)} mutant littermates at postnatal day 1 and day 75 (P1 and P75), respectively. (FIG. 2B-2C) Quantitation of brain weights from CASK^{(+/+)} and CASK^{(+/-)} mice at P1 and P75 (* indicates p<0.05;n=4). (FIG. 2D) H&E stained sections of the cerebellum at P75 showing pronounced cerebellar hypoplasia in CASK^{(+/+)} mice relative to the (ASK (-/-) control. (FIG. 2E) Area of cerebellar sections obtained from CASK (+/-) and CASK^{(+/-)} mice. Measurements were made using ImageJ software and normalized to sex-matched wild-type littermate controls Bar graphs are plotted as mean±SEM; (* indicates p<0.05; n=4). (FIG. 2F) High magnification of the indicated square regions from panel D showing relatively fewer cells in the IGL of cerebellar folia of CASK^{(+/-)} mice compared to the CASK^{(+/+)} control. (FIG. 2G) Cerebella from P5 mouse pups of indicated genotype were dissected and fixed in PFA. Parasaggital sections were stained with a green nucleic acid stain to reveal the external granular layer (EGL). (FIG. 2H) Quantitation of thickness of EGL is plotted as mean±SEM; n=3.

FIG. 3A-3F—CASK haploinsufficiency promotes changes in mitochondrial molecule and metabolism in the brain. (FIG. 3A) iTraq quantitative proteomic data were analyzed. 525proteins, excluding CASK, are altered in the brain of CASK+/−mice compared to wildtype female littermates. ProfCom analysis on the data is provided (Antonov, Schmidt et al., 2008). Mitochondrion is at the top of the list. (FIG. 3B) Basal oxygen consumption rate measured in brain homogenates isolated from one-month-old CASK(+/+) and CASK(+/−) mice. Respiration rate is normalized to total protein content. (FIG. 3C) Glucose oxidation rate measured in brain homogenates isolated from CASK(+/+) and CASK(+/−) sex-matched littermate mice. (FIG. 3D) Palmitate oxidation rate measured in brain homogenates isolated from CASK(+/+) and CASK(+/−) sex-matched littermate mice. (FIG. 3E) Measurement of arachidonic acid abundance using mass spectrometry from brain homogenates isolated from CASK(+/+) and CASK(+/−) sex-matched littermate mice. (FIG. 3F) Measurement of ROS in brain homogenates isolated from CASK(+/+) and CASK(+/−) sex-matched littermate mice. (* indicates p<0.05; n=4).

FIG. 4A-4G—Deletion of Cask from post-migratory cerebellar cells results in profound cerebellar degeneration. (FIG. 4A) Gross images of age-matched Cask^{floxed} control and (FIG. 4B) Cask^{floxed}; Calb2-Cre brains after plateau of ataxia; arrow indicates diminished volume of the cerebellum while the remainder of the brain remains similarly sized. (FIG. 4C) 10× image of Cask^{floxed}; Calb2-Cre at P30 (top) and P100 (bottom), demonstrating severely reduced cross-sectional area, molecular layer width and granular layer width at P100; the arrow indicates expanded white matter; the bracket indicates diminished overall cerebellar size. (FIGS. 4D-4F) Quantification of the entire cross-sectional area pre-and post-ataxia, molecular layer width, and granular layer width. (FIG. 4G) Ratio of white matter area to total cross-sectional area pre- and post-ataxia. n=3 for (FIGS. 4B-4G). Despite increase in width of white matter area, we found a trend towards decrease in myelin (Patel, Hegert et al., 2021), indicating this area is made up only of extracellular matrix.

FIG. 5A-5B.-Blocking mitochondrial ROS protects cerebellum from degenerating in absence of CASK. (FIG. 5A) CASK^{floxed}; Calb2-cre mice do not express CASK in cerebellum beyond P15 (post-natal day 15). Cerebellum undergoes atrophy after P30. Images of cerebellum from CASK^{floxed}; Calb2-cre mice on P30 and P100 shows the cerebellar atrophy (left and middle panel). Treatment with SkQ1 leads to rescue of cerebellar atrophy (right panel). (FIG. 5B) Cerebellum undergoes atrophy in CASK^{floxed}; Calb2-cre mice leading to locomotor incoordination. The data represents latency time to fall in an accelerating rotarod experiment on P70. CASK^{floxed}. Calb2-cre mice without treatment cannot balance on rotarod, but CASK^{floxed}; Calb2-cre mice treated with SkQ1 balance is completely rescued. Data represents mean and SEM from n=3. * indicates p≤ 0.05.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub- ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W. H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th Ed.} 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall, Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g., a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “derivative” can refer to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

As used herein, “gene product” refers to any polynucleotide, polypeptide, and/or the like that is ultimately produced from transcribing a gene and optionally translating the transcript.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, the term “CASK-linked neurodevelopmental disorder” refers to disorders, diseases, and conditions, etc. of the central nervous system and/or peripheral nervous system, including symptoms and secondary diseases, disorders, and/or conditions, that result from a dysfunctional Cask gene where the expression of and/or production of a Cask gene product is reduced or eliminated as compared to a normal and/or wild-type Cask gene.

As used herein, “control” refers to an alternative subject or sample used in an experiment for comparison purpose and included to minimize or distinguish the effect of variables other than an independent variable.

As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, the suitable control is a sample from a healthy individual or otherwise normal individual.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Pontocerebellar hypoplasias (PCH) are a group of disorders characterized by a thin cerebellum and brain stem. PCH are neurodegenerative conditions that begin prenatally and have a poor prognosis. Overall, there have been very few investigations into the mechanism of PCH, and there are no existing therapies.

Mutations in the X-linked gene CASK are very commonly associated with PCH. Applicant has demonstrated that cellular and molecular mechanisms of CASK-linked PCH are similar to other forms of PCH. Using studies in human subjects and murine models, Applicant has determined that loss of CASK affects mitochondrial metabolism and increases ROS (reactive oxygen species) production causing PCH. Blocking mitochondrial ROS prevents this PCH. Administration of mitochondrial antioxidant SkQ1 effectively inhibits neurodegeneration resulting from CASK-loss, suggesting that SkQ1 and other SkQ compounds can be beneficial therapeutic drug for at least PCH. CASK variants have been found in some children diagnosed with Rett syndrome, suggesting that SkQ compounds may also be beneficial in a subset of Rett syndrome cases or other diseases whose pathology involves CASK-loss. Described in certain embodiments herein are methods of treatment for PCH and other diseases in which CASK-loss plays a role in disease pathology or symptoms that includes administering one or more SkQ compounds to the subject in need thereof. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Treating Cask-Linked Neurodevelopmental Disorders

Described in certain example embodiments herein are methods of treating a CASK-linked neurodevelopmental disorder and/or a symptom thereof in a subject in need thereof, the method comprising administering an amount of an SkQ compound or a variant or derivative thereof or formulation thereof to the subject in need thereof. kQ molecules are a family of small, positively charged molecules that act as mitochondria-targeted antioxidants. The “Sk” in SkQ stands for Skulachev, while the “Q” refers to the molecule's resemblance to coenzyme Q, a molecule that is essential for energy production in mitochondria. SkQ molecules are designed to penetrate the cell membrane and accumulate specifically in mitochondria, where they can neutralize harmful reactive oxygen species (ROS) and prevent oxidative damage. SkQ molecules are unique in that they are specifically targeted to mitochondria, which are the primary source of ROS in cells.

In certain example embodiments, the SkQ compound is selected from SkQ1 (10-(6′-Plastoquinonyl)decyltriphenylphosphonium), SkQR1 (10-(6′-Plastoquinonyl)decylrhodamine-19), SkQ2 (10-(6′-plastoquinonyl)decylcarnitine), SkQ2M (10-(6′-plastoquinonyl)decylmethylcarnitine), SkQ3 (10-(6′-methylplastoquinonyl) decyltriphenylphosphonium), SkQ4 (10-(6′-plastoquinonyl)decyltributylammonium), SkQ5 (5-(6′-plastoquinonyl)amyltriphenylphosphonium), SkqBerb (13-[9-(6-plastoquinonyl) nonyloxycarbonyl-methyl]berberine), SkQPalm (13-[9-(6-plastoquinonyl)nonyloxycarbonyl-methyl]palmatine), Ct12TPP (dodecyltriphenylphosphonium), MitoQ (10-(6-ubiquinoyl)decyltriphenyl-phosphonium) or a derivative thereof, and any combination thereof. In some embodiments, a pharmaceutical formulation containing one or more SkQ compounds, variant(s) thereof, derivative(s) thereof, or any combination thereof described herein is administred to the subject in need thereof.

The amount of the SkQ compound, variant thereof, derivative thereof, etc. administered can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

The concentration of the SkQ compound, variant thereof, derivative thereof, etc. administered can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

The amount of the SkQ compound, variant thereof, derivative thereof, etc. administered can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

The SkQ compound can be administered by any suitable route. In some embodiments, administration is oral. In some embodiments, administration is directly into the central nervous system. In some embodiments, administration is intracerebroventricular, intrathecal, or intranasal administration.

In certain example embodiments, the CASK-linked neurodevelopmental disorder and/or a symptom thereof is a cerebellar hypoplasia. In certain example embodiments, the CASK-linked neurodevelopmental disorder and/or a symptom thereof is a Pontocerebellar hypoplasia (PHC). In certain example embodiments, the PHC is a PHC type 1, a PHC type 2, PHC type 3,PHC type 4, PHC type 5, or PHC type 6. In certain example embodiments, the PHC type 1 is PHC type 1A or PHC type 1B. In certain example embodiments, the PHC type 2 is PHC type 2A, PHC type 2B, or PHC type 2C. In certain example embodiments, the CASK-linked neurodevelopmental disorder and/or a symptom thereof is microcephaly with pontine and cerebellar hypoplasia. In certain example embodiments, the CASK-linked neurodevelopmental disorder and/or a symptom thereof is an X-linked intellectual disability. In certain example embodiments, the CASK-linked neurodevelopmental disorder and/or a symptom thereof is epilepsy. In certain example embodiments, the CASK-linked neurodevelopmental disorder and Rett syndrome.

In certain example embodiments, mitochondrial metabolism is improved in the subject in need thereof as measured by a decrease in brain reactive oxygen species in the brain, an increase in brain mitochondrial basal respiration, increase in brain glucose oxidation, a decrease in brain lipid oxidation, an increase in the levels of brain arachidonic acid as compared to the subject in need thereof prior to administration or other suitable control or threshold value.

In certain example embodiments, neuron loss, neuron dysfunction, neurodegeneration, or any combination thereof is decreased in the subject in need thereof after administration as compared to the subject in need thereof prior to administration or other suitable control or threshold value.

In some embodiments, mitochondrial metabolism, particularly in the brain and/or central nervous system (CNS) in the subject in need thereof is improved as measured by a decrease in brain reactive oxygen species in the brain, an increase in brain mitochondrial basal respiration, increase in brain glucose oxidation, a decrease in brain lipid oxidation, an increase in the levels of brain arachidonic acid as compared to the subject in need thereof prior to administration or other suitable control or threshold value. In some embodiments, brain reactive oxygen species in the subject in need thereof is decreased 1-1000 fold or more. In some embodiments, brain glucose oxidation is increased 1-1000 fold or more in the subject in need thereof. In some embodiments, brain lipid oxidation in the subject in need thereof is decreased 1-1000 fold or more. In some embodiments, the levels of brain arachidonic acid in the subject in need thereof is increased 1-1000 fold or more.

In some embodiments, neuron loss, neuron dysfunction, neurodegeneration, or any combination thereof in the subject in need thereof is decreased after administration as compared to the subject in need thereof prior to administration or other suitable control or threshold value. In some embodiments, neuron loss, neuron dysfunction, neurodegeneration, or any combination thereof in the subject in need thereof is decreased 1-1000 fold or more after administration as compared to the subject in need thereof prior to administration or other suitable control or threshold value.

The SkQ compound(s) variant(s) thereof, derivative(s) thereof, and/or formulations thereof can be administered to the subject in need thereof one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, The SkQ compound(s) variant(s) thereof, derivative(s) thereof, and/or formulations thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively.

In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation. The method of claim 1, wherein administration of the SkQ compound or formulation thereof occurs during a therapeutic temporal window.

In some embodiments, one or more co-therapies or co-treatments are delivered or provided to the subject in need thereof in addition to the SkQ compound(s) variant(s) thereof, derivative(s) thereof, and/or formulations thereof. Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time. In some embodiments, the co-therapy or co-treatment includes administering a lipid peroxidation to the subject in need thereof. Exemplary lipid peroxidation inhibitors are described elsewhere herein in connection with secondary active agents below.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, one or more SkQ compounds, variants thereof, derivatives thereof, or any combination thereof. In some embodiments, the SkQ compound is selected from SkQ1 (10-(6′-Plastoquinonyl)decyltriphenylphosphonium), SkQR1 (10-(6′-Plastoquinonyl)decylrhodamine-19), SkQ2 (10-(6′-plastoquinonyl)decylcarnitine), SkQ2M (10-(6′-plastoquinonyl)decylmethylcarnitine), SkQ3 (10-(6′-methylplastoquinonyl) decyltriphenylphosphonium), SkQ4 (10-(6′-plastoquinonyl)decyltributylammonium), SkQ5 (5-(6′-plastoquinonyl)amyltriphenylphosphonium), SkqBerb (13-[9-(6-plastoquinonyl) nonyloxycarbonyl-methyl]berberine), SkQPalm (13-[9-(6-plastoquinonyl)nonyloxycarbonyl-methyl]palmatine), Ct12TPP (dodecyltriphenylphosphonium), MitoQ (10-(6-ubiquinoyl)decyltriphenyl-phosphonium) or a derivative thereof, and any combination thereof.

In some embodiments, the active ingredient (e.g., an SkQ compound) is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a CASK-linked neruodisease. In certain example embodiments, the CASK-linked neurodevelopmental disorder and/or a symptom thereof is a cerebellar hypoplasia. In certain example embodiments, the CASK-linked neurodevelopmental disorder and/or a symptom thereof is a Pontocerebellar hypoplasia (PHC). In certain example embodiments, the PHC is a PHC type 1, a PHC type 2, PHC type 3, PHC type 4, PHC type 5, or PHC type 6. In certain example embodiments, the PHC type 1 is PHC type 1A or PHC type 1B. In certain example embodiments, the PHC type 2 is PHC type 2A, PHC type 2B, or PHC type 2C. In certain example embodiments, the CASK-linked neurodevelopmental disorder and/or a symptom thereof is microcephaly with pontine and cerebellar hypoplasia. In certain example embodiments, the CASK-linked neurodevelopmental disorder and/or a symptom thereof is an X-linked intellectual disability. In certain example embodiments, the CASK-linked neurodevelopmental disorder and/or a symptom thereof is epilepsy. In certain example embodiments, the CASK-linked neurodevelopmental disorder and Rett syndrome. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatoi anti-histamines, anti-infectives, chemotherapeutics, and combinations thereof.

In some embodiments, the secondary active agent is a lipid peroxidation inhibitor. Exemplary lipid peroxidation inhibitors include, but are not limited to antioxidants (e.g., vitamin E, vitamin C, and beta-carotene), phenolic compounds (e.g., resveratrol, curcumin, and quercetin), synthetic antioxidants (e.g., butylated hydroxyanisole, butylated hydroxytoluene), Omega-3 fatty acids (Docosahexaenoic acid (DHA) and eicosapentaenoic acid (EPA)), and ADA-409-052. In some embodiments, the secondary active agent is ADA-409-052.

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects are or include an improvement in mitochondrial metabolism, particularly in the brain and/or central nervous system (CNS) in the subject in need thereof as measured by a decrease in brain reactive oxygen species in the brain, an increase in brain mitochondrial basal respiration, increase in brain glucose oxidation, a decrease in brain lipid oxidation, an increase in the levels of brain arachidonic acid as compared to the subject in need thereof prior to administration or other suitable control or threshold value. In some embodiments, the therapeutic effect is a 1-1000 fold or more decrease in brain reactive oxygen species in the subject in need thereof. In some embodiments, the therapeutic effect is a 1-1000 fold or more increase in brain glucose oxidation in the subject in need thereof. In some embodiments, the therapeutic effect is a 1-1000 fold or more decrease in brain lipid oxidation in the subject in need thereof. In some embodiments, the therapeutic effect is a 1-1000 fold or more an increase in the levels of brain arachidonic acid in the subject in need thereof.

In some embodiments, the one or more therapeutic effects are or include a decrease in neuron loss, neuron dysfunction, neurodegeneration, or any combination thereof in the subject in need thereof after administration as compared to the subject in need thereof prior to administration or other suitable control or threshold value. In some embodiments, the one or more therapeutic effects are or include a 1-1000 fold or more decrease in neuron loss, neuron dysfunction, neurodegeneration, or any combination thereof in the subject in need thereof after administration as compared to the subject in need thereof prior to administration or other suitable control or threshold value.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non- aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof. In some embodiments, the subject in need thereof has or is suspected of having a CASK-linked neurodevelopmental disorder. In certain example embodiments, the CASK-linked neurodevelopmental disorder and/or a symptom thereof is a cerebellar hypoplasia. In certain example embodiments, the CASK-linked neurodevelopmental disorder and/or a symptom thereof is a Pontocerebellar hypoplasia (PHC). In certain example embodiments, the PHC is a PHC type 1, a PHC type 2, PHC type 3, PHC type 4, PHC type 5, or PHC type 6. In certain example embodiments, the PHC type 1 is PHC type 1A or PHC type 1B. In certain example embodiments, the PHC type 2 is PHC type 2A, PHC type 2B, or PHC type 2C. In certain example embodiments, the CASK-linked neurodevelopmental disorder and/or a symptom thereof is microcephaly with pontine and cerebellar hypoplasia. In certain example embodiments, the CASK-linked neurodevelopmental disorder and/or a symptom thereof is an X-linked intellectual disability. In certain example embodiments, the CASK-linked neurodevelopmental disorder and/or a symptom thereof is epilepsy. In certain example embodiments, the CASK-linked neurodevelopmental disorder and Rett syndrome.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in° C., and pressure is at or near atmospheric. Standard temperature and pressure are defined as 20° C. and 1 atmosphere.

### Example 1

### Introduction

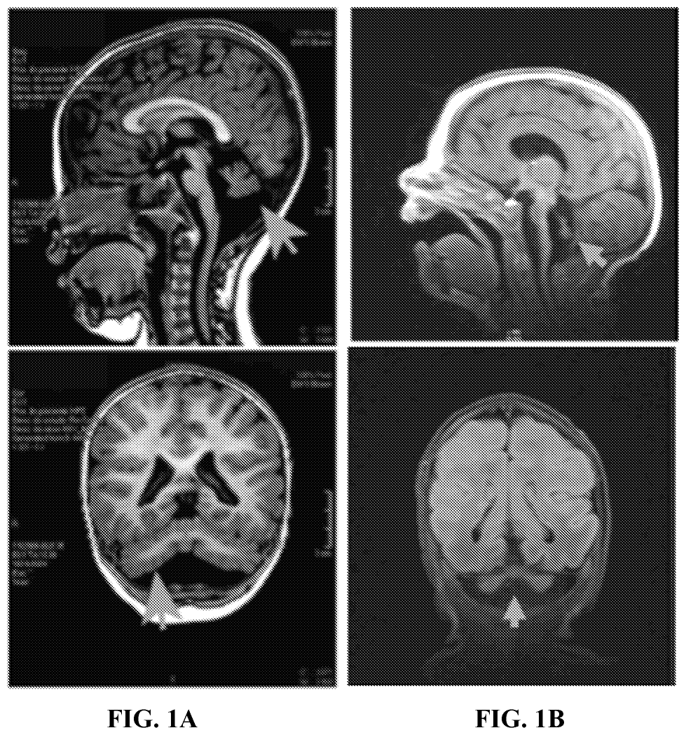

PCH (pontocerebellar hypoplasia) are clinically and genetically heterogeneous group of pediatric neurological disorders characterized by a thin brain stem and cerebellum with progressive microcephaly (Namavar, Barth et al., 2011, van Dijk, Baas et al., 2018). Severe psychomotor and cognitive disabilities and even seizures are reported. In most cases, PCH are autosomal in nature, and the clinical course is downhill with a poor prognosis with death occurring in infancy or early childhood (Namavar et al., 2011, van Dijk et al., 2018). At present, there are no therapeutic drugs available for this group of disorders. Mutations in the ubiquitously expressed gene CASK (calcium/calmodulin-dependent serine protein kinase) (Hata, Butz et al., 1996, Stevenson, Laverty et al., 2000) has also been associated with PCH and in some series been found to be one of the most prevalent cause of PCH (Burglen, Chantot-Bastaraud et al., 2012, Nuovo, Micalizzi et al., 2021, Valayannopoulos, Michot et al., 2012) (FIG. 1A-1B), but due to its non-progressive nature and absence of supratentorial findings it has been considered to be a separate entity by some investigators (van Dijk et al., 2018, van Dijk, Barth et al., 2021). Mutations in CASK gene lead to microcephaly and global developmental delay, with a disproportionate impact on the brainstem and cerebellum (Burglen et al., 2012, Moog, Bierhals et al., 2015, Moog, Kutsche et al., 2011, Najm, Horn et al., 2008). CASK is an X-linked gene with two copies present in females and only one copy in males, therefore expression pattern of mutant CASK gene is different than those of autosomal gene. Much like autosomal PCH, loss of CASK function produces epilepsy, supratentorial atrophy with severe PCH and is often lethal in males due to central respiratory failure (Mukherjee, Patel et al., 2020, Saitsu, Kato et al., 2012) (FIG. 1A-1B). Heterozygous loss of CASK gene in females produces a condition called microcephaly with pontine and cerebellar hypoplasia (MICPCH). MICPCH involves postnatal progressive microcephaly similar to other PCH (Burglen et al., 2012, Moog et al., 2011). Through a series of published and unpublished studies, Applicant established that MICPCH is not neurodevelopmental in origin (as assumed (van Dijk et al., 2021)), but that loss of CASK produces neurodegeneration which disproportionately affects the hindbrain including cerebellum (Liang, Kerr et al., 2017, Srivastava, McMillan et al., 2016). Overall, therefore CASK mutant mice serves as a good model for PCH and provides us with the opportunity to uncover underlying mechanisms and test therapeutic strategies to ameliorate PCH. Applicant has so far uncovered that CASK loss produces postnatal neuronal loss and that there is an increase in reactive oxygen species (ROS) secondary to mitochondrial dysfunction (Kerr, Patel et al., 2019, Patel, Liang et al., 2020, Srivastava et al., 2016). Applicant also has evidence that abolishing mitochondrial reactive oxygen species (ROS) can prevent cerebellar degeneration produced by CASK deficit.

### Results

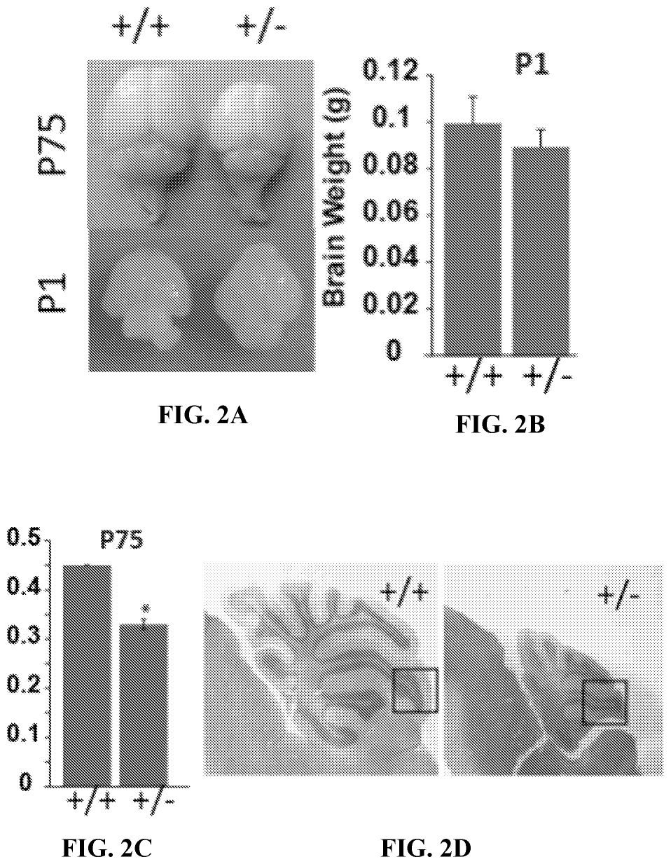

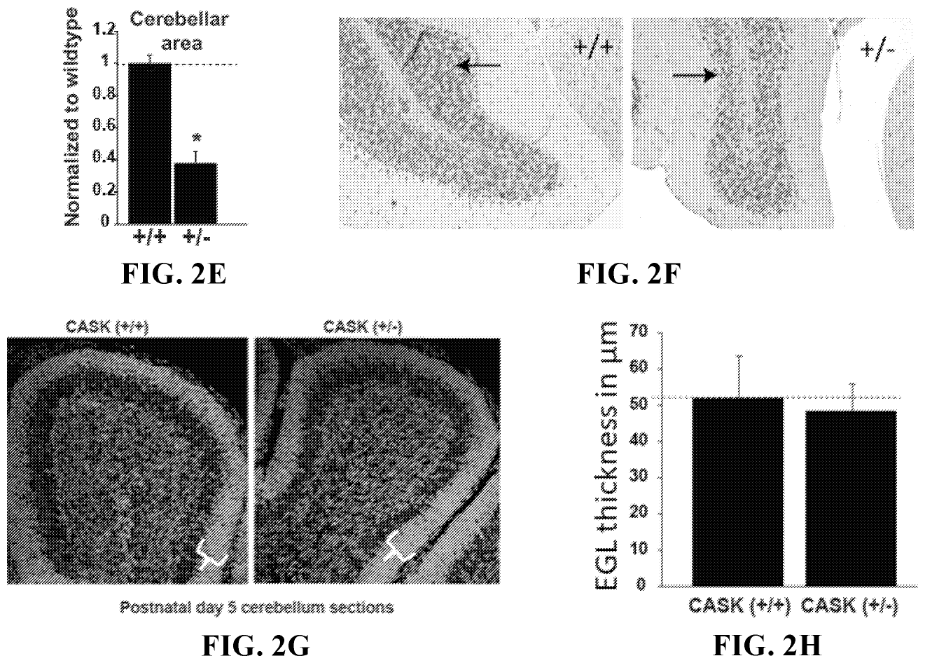

Applicant first examined if it was at all possible to recapitulate the MICPCH phenotype using a murine model. Applicant designed and produced CASK heterozygous knockout mice similar to heterozygous female population of MICPCH. Due to the X-linkage, this study was confined to female animals. The CASK^{+/-} mice recapitulated many of the features of MICPCH, including postnatal microcephaly, disproportionate cerebellar hypoplasia, optic nerve hypoplasia, scoliosis, and even sporadic seizures ((Liang et al., 2017, Patel et al., 2020, Srivastava et al., 2016) and FIG. 2A-2H. The scoliosis, most likely resulted from central hypotonia. Multiple locomotor defects including hyperkinesia, loss of coordination and hind-limb clasping reflex were uncovered (Kerr et al., 2019, Srivastava et al., 2016). Applicant examined the electrical activity of the CASK^{+/-} mouse cortex and retina using EEG and ERG (electroretinogram) and found it to be indistinguishable from wildtype littermates (Kerr et al., 2019, Patel et al., 2020).

Applicant also confirmed that CASK loss does not selectively affect cerebellar development. First, Applicant deleted CASK from Purkinje cells in the developing cerebellum specifically. This manipulation did not affect either the survival of Purkinje cells or locomotor function of the mice during the period of observation (Srivastava et al., 2016). Then, to test whether deletion of CASK had any effect on maturation and migration of granule cells of the cerebellum, Applicant deleted CASK from a subset of granule cell precursors within the rhombic lip. Applicant did not observed any change in the trajectory of development or migration of the granule cells that lacked CASK (Srivastava et al., 2016). These data were consistent with Applicant's finding that although CASK+/−mice display a reduced number of neurons in the inner granule layer (IGL), the external granular layer (EGL; where granule cells mature) remains unperturbed during the developmental phase (Srivastava et al., 2016) (FIG. 2G and 2H). These findings led us to conclude that CASK loss does not affect neuronal development. Indeed, both the brain and optic nerve in mice show a size reduction compared to littermates around one week after they are born (Liang et al., 2017, Srivastava et al., 2016).

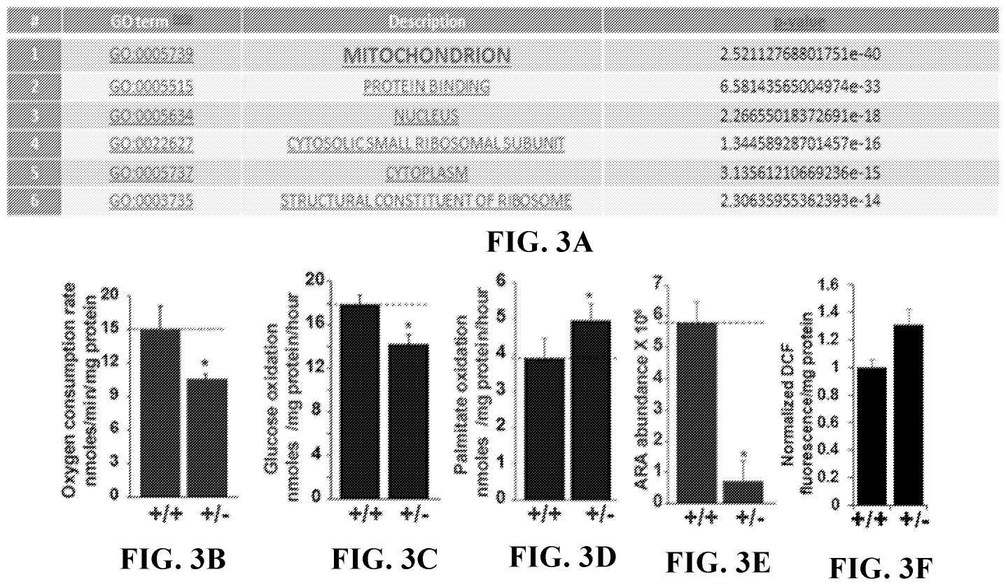

CASK exists as part of larger protein complexes (Srivastava et al., 2016). To fully describe the protein complexes associated with CASK, Applicant has performed extensive immunoprecipitation experiments. First, Applicant performed such experiments in the fly model and were able to confirm many of the previously known CASK interactions, including with Mint, veli, neurexin, and CaMKII (Mukherjee, Slawson et al., 2014). Surprisingly, Applicant found that CASK likely interacts with different proteins in different neurons, allowing for functional diversification of CASK. A common set of proteins that immunoprecipitated with CASK were mitochondrial (Mukherjee et al., 2014). Applicant also performed immunoprecipitation from the rat brain and found that CASK was part of four distinct complexes: 1) synaptic, 2) ribosomal/protein chaperone, 3) cytoskeletal and 4) mitochondrial (Patel et al., 2020). CASK interactions with mitochondrial proteins may thus be conserved in different animal models. Applicant next tested whether these interactions have any physiological relevance. Applicant examined transcriptomic changes in the CASK+/−mouse brain using an RNAseq approach; only about 100 transcripts exhibited significant changes, of which many were extracellular matrix (ECM) molecules. Changes in the ECM are noted in other neurodegenerative conditions (Bonneh-Barkay & Wiley, 2009) and may reflect ECM turnover. Applicant has also examined changes in protein levels using quantitative proteomics. More than 500 proteins are differentially expressed in CASK+/− mice. Interestingly, these differentially expressed proteins fall into the same categories occupied by the proteins with which CASK forms complexes (Patel et al., 2020). Ninety-nine of the proteins belong to the mitochondrial group, affirming Applicant's earlier findings of a link between CASK and the mitochondrion and suggesting that CASK regulates mitochondrial function (Patel et al., 2020) (FIG. 3A). KEGG (Kyoto Encyclopedia of Genetics and Genomics) pathway analysis of protein changes with CASK loss indicates that the highest significance is associated with neurodegenerative conditions and the metabolic pathway of oxidative phosphorylation (Mukherjee, LaConte et al., 2022).

Based on the molecular data Applicant next tested the CASK^{-/-} brain mitochondrial metabolism. Consistent with the molecular changes Applicant observed a decrease in brain mitochondrial basal respiration. Applicant also uncovered that in CASK^{+/-} brain glucose oxidation is reduced and lipid oxidation is increased. Due to increased electron leak such shift in brain metabolism is likely to increase in mitochondrial reactive oxygen species (ROS) generation (Speijer, Manjeri et al., 2014). Indeed, Applicant observed an increase in overall ROS in brain homogenates from CASK^{+/-} mice. Along with that Applicant also observed a decrease in the levels of a polyunsaturated fatty acid, arachidonic acid in the brain homogenate. A decrease in arachidonic acid is typically associated with ROS dependent cell-death (Magtanong, Ko et al., 2016) (FIG. 3B-3F).

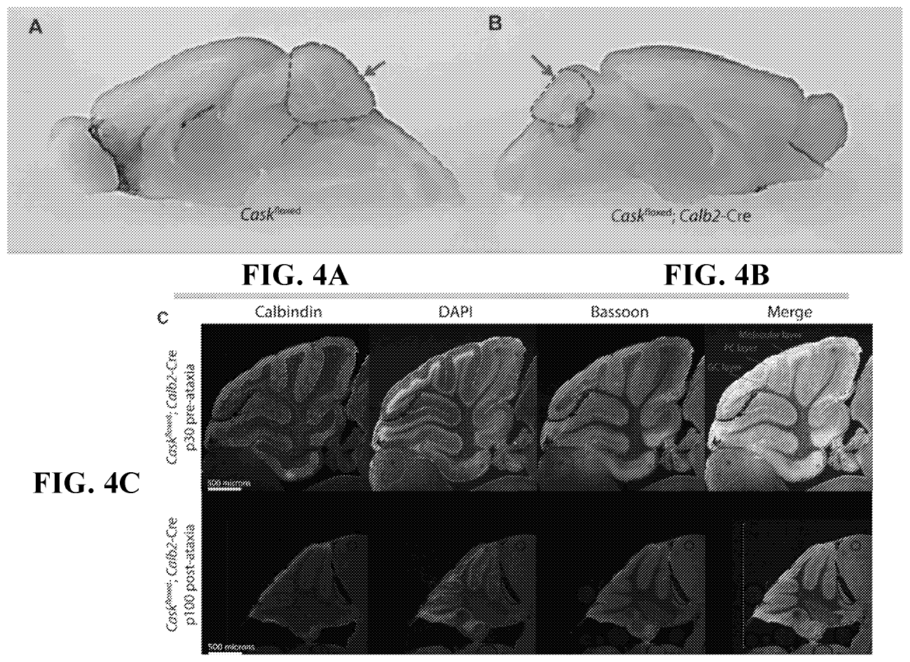

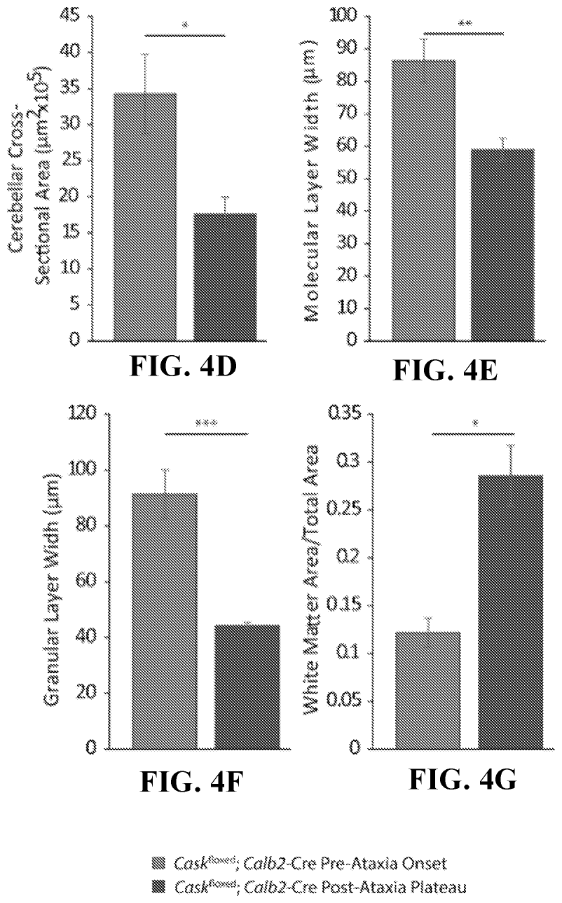

With no evidence for a defect in neuronal development and biochemical evidence for mitochondrial ROS mediated neuronal death, Applicant next decided to determine if CASK loss results in the degeneration of formed neurons. In the human brain without CASK, Applicant observed astrogliosis as well as microgliosis, both hallmarks of neuronal damage and neuronal loss (Patel, Hegert et al., 2022). In the setting of a human brain without CASK, the presumptive neuronal loss occurs during the early development of the cerebellum, making it hard to further interpret the results. To better address the question of degeneration in the setting of early development, Applicant next synchronized the timing of CASK deletion with the completion of neuronal migration in the cerebella of mice by crossing CASK^{floxed} mice (stock #006382) with a mouse line expressing Cre recombinase only in post-migratory cerebellar neurons (postnatal day 15 (P15)) under endogenous calretinin promoter (stock #010774) (Patel et al., 2021). Applicant observed that this manipulation leads to rapid atrophy of the cerebellum, specifically of the granule cell layer, beginning at 2 months (Patel et al., 2021) (FIG. 4A-4G). The mice remain otherwise healthy but lose their balance completely (Patel et al., 2022). Interestingly, the loss of balance does not occur until cerebellar atrophy is present, indicating that the observed ataxia results from damage to the tissue rather than loss of CASK molecular function. Applicant's careful study of CASK mice overall thus suggests that loss of CASK induces pathology in the form of neuronal injury and neuronal death and that behavioral phenotypes are most likely secondary to structural changes. These changes are associated with mitochondrial dysfunction.

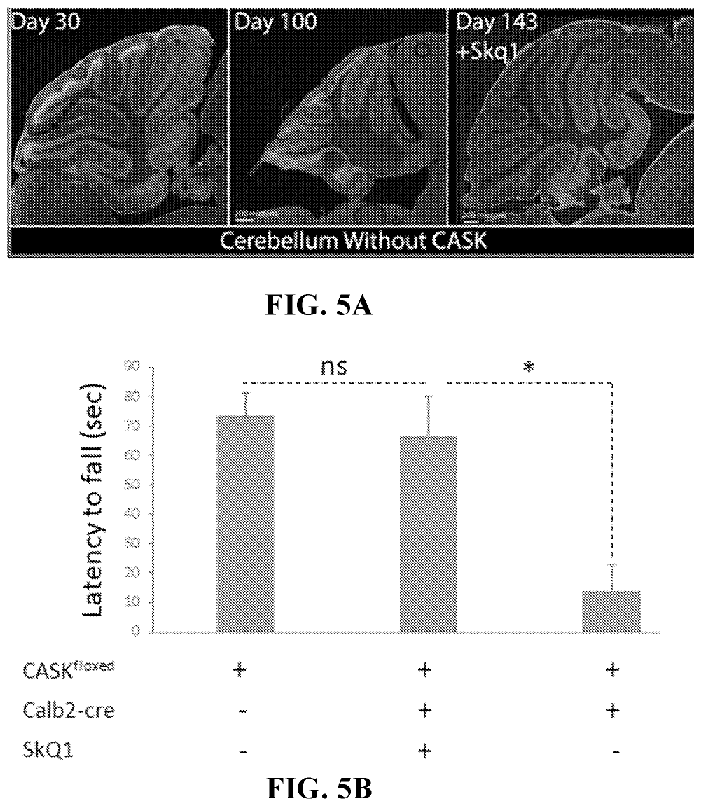

Cerebellum is disproportionately affected in several mitochondrial and metabolic disorders (Lax, Hepplewhite et al., 2012, Liu, Li et al., 2016, Scaglia, Wong et al., 2005, Silver & Mercimek-Andrews, 2020, Steinlin, Blaser et al., 1998). PCH1 and PCH6 have been also linked with mitochondrial defects, and mutations in mitochondrial molecules like RARS2, ATAD3 and SLC25A46 are associated with PCH (Desai, Frazier et al., 2017, van Dijk, Rudnik-Schoneborn et al., 2017, van Dijk, van Ruissen et al., 2017). Further some non-mitochondrial PCH-linked genes like EXOSC3 and VRK1 may also participate in mitochondrial and metabolic pathways (Park, Artan et al., 2020, Schottmann, Picker-Minh et al., 2017). Thus, beyond mitochondrial defect may be one of the major pathway that is disrupted in PCH. Applicant's data suggest that, like some other models of cerebellar hypoplasia and granule cell degeneration, CASK deletion is also likely to profoundly affect mitochondrial ROS. Applicant tested the effect of SkQ1, a mitochondrially targeted antioxidant on cerebellar degeneration caused by CASK deletion in cerebellum of CASKfloxed; Calb2-cre mice. Applicant's data indicate that SkQ1 can halt CASK-linked cerebellar degeneration (FIG. 5A-5B). Based on the data described here and without being bound by theory Applicant hypothesizes that the administration of mitochondrial-targeted ROS inhibitors will rescue neurological manifestations of CASK-linked and other PCH.

### References for Example 1

Antonov AV, Schmidt T, Wang Y, Mewes HW (2008) ProfCom: a web tool for profiling the complex functionality of gene groups identified from high-throughput data. Nucleic Acids Res 36: W347-W351
Bonneh-Barkay D, Wiley CA (2009) Brain extracellular matrix in neurodegeneration. Brain Pathol 19:573-85
Burglen L, Chantot-Bastaraud S, Garel C, Milh M, Touraine R, Zanni G, Petit F, Afenjar A, Goizet C, Barresi S, Coussement A, Ioos C, Lazaro L, Joriot S, Desguerre I, Lacombe D, des Portes V, Bertini E, Siffroi JP, de Villemeur TB et al. (2012) Spectrum of pontocerebellar hypoplasia in 13 girls and boys with CASK mutations: confirmation of a recognizable phenotype and first description of a male mosaic patient. Orphanet J Rare Dis 7:18
Desai R, Frazier A E, Durigon R, Patel H, Jones A W, Dalla Rosa I, Lake N J, Compton A G, Mountford H S, Tucker E J, Mitchell A L R, Jackson D, Sesay A, Di Re M, van den Heuvel L P, Burke D, Francis D, Lunke S, McGillivray G, Mandelstam S et al. (2017) ATAD3 gene cluster deletions cause cerebellar dysfunction associated with altered mitochondrial DNA and cholesterol metabolism. Brain 140:1595-1610
Hata Y, Butz S, Sudhof T C (1996) CASK: a novel dlg/PSD95 homolog with an N-terminal calmodulin-dependent protein kinase domain identified by interaction with neurexins. J Neurosci 16:2488-94
Kerr A, Patel P A, LaConte L E W, Liang C, Chen C K, Shah V, Fox M A, Mukherjee K (2019) Non-Cell Autonomous Roles for CASK in Optic Nerve Hypoplasia. Invest Ophthalmol Vis Sci 60:3584-3594
Lax N Z, Hepplewhite P D, Reeve A K, Nesbitt V, McFarland R, Jaros E, Taylor R W, Turnbull D M (2012) Cerebellar ataxia in patients with mitochondrial DNA disease: a molecular clinicopathological study. J Neuropathol Exp Neurol 71:148-61
Liang C, Kerr A, Qiu Y F Z, Cristofoli F, Van Esch H, Fox M A, Mukherjee K (2017) Optic Nerve Hypoplasia Is a Pervasive Subcortical Pathology of Visual System in Neonates. Invest Ophth Vis Sci 58:5485-5496
Liu C, Li X, Lu B (2016) The Immp21 mutation causes age-dependent degeneration of cerebellar granule neurons prevented by antioxidant treatment. Aging Cell 15:167-76
Magtanong L, Ko PJ, Dixon SJ (2016) Emerging roles for lipids in non-apoptotic cell death. Cell Death Differ 23:1099-109
Moog U, Bierhals T, Brand K, Bautsch J, Biskup S, Brune T, Denecke J, de Die-Smulders CE, Evers C, Hempel M, Henneke M, Yntema H, Menten B, Pietz J, Pfundt R, Schmidtke J, Steinemann D, Stumpel CT, Van Maldergem L, Kutsche K (2015) Phenotypic and molecular insights into CASK-related disorders in males. Orphanet J Rare Dis 10:44
Moog U, Kutsche K, Kortum F, Chilian B, Bierhals T, Apeshiotis N, Balg S, Chassaing N, Coubes C, Das S, Engels H, Van Esch H, Grasshoff U, Heise M, Isidor B, Jarvis J, Koehler U, Martin T, Oehl-Jaschkowitz B, Ortibus E et al. (2011) Phenotypic spectrum associated with CASK loss-of-function mutations. J Med Genet 48:741-51
Mukherjee K, LaConte LEW, Srivastava S (2022) The Non-Linear Path from Gene Dysfunction to Genetic Disease: Lessons from the MICPCH Mouse Model. Cells 11
Mukherjee K, Patel PA, Rajan DS, LaConte LEW, Srivastava S (2020) Survival of a male patient harboring CASK Arg27Ter mutation to adolescence. Mol Genet Genomic Med: e1426
Mukherjee K, Slawson J B, Christmann BL, Griffith LC (2014) Neuron-specific protein interactions of Drosophila CASK-beta are revealed by mass spectrometry. Front Mol Neurosci 7:58
Najm J, Horn D, Wimplinger I, Golden JA, Chizhikov VV, Sudi J, Christian SL, Ullmann R, Kuechler A, Haas CA, Flubacher A, Charnas LR, Uyanik G, Frank U, Klopocki E, Dobyns WB, Kutsche K (2008) Mutations of CASK cause an X-linked brain malformation phenotype with microcephaly and hypoplasia of the brainstem and cerebellum. Nat Genet 40:1065- 7
Namavar Y, Barth PG, Poll-The BT, Baas F (2011) Classification, diagnosis and potential mechanisms in pontocerebellar hypoplasia. Orphanet J Rare Dis 6:50
Nuovo S, Micalizzi A, Romaniello R, Arrigoni F, Ginevrino M, Casella A, Serpieri V, D'Arrigo S, Briguglio M, Salerno GG, Rossato S, Sartori S, Leuzzi V, Battini R, Ben-Zeev B, Graziano C, Mirabelli Badenier M, Brankovic V, Nardocci N, Spiegel R et al. (2021) Refining the mutational spectrum and gene-phenotype correlates in pontocerebellar hypoplasia: results of a multicentric study. J Med Genet
Park S, Artan M, Han SH, Park HH, Jung Y, Hwang AB, Shin WS, Kim KT, Lee SV (2020) VRK-1 extends life span by activation of AMPK via phosphorylation. Sci Adv 6
Patel PA, Hegert J, Cristian I, Kerr A, LaConte LE, Fox MA, Srivastava S, Mukherjee K (2021) Complete loss of CASK causes severe ataxia through cerebellar degeneration in human and mouse. bioRxiv
Patel PA, Hegert JV, Cristian I, Kerr A, LaConte LEW, Fox MA, Srivastava S, Mukherjee K (2022) Complete loss of the X-linked gene CASK causes severe cerebellar degeneration. J Med Genet
Patel PA, Liang C, Arora A, Vijayan S, Ahuja S, Wagley PK, Settlage R, LaConte LEW, Goodkin HP, Lazar I, Srivastava S, Mukherjee K (2020) Haploinsufficiency of X-linked intellectual disability gene CASK induces post-transcriptional changes in synaptic and cellular metabolic pathways. Exp Neurol 329:113319
Saitsu H, Kato M, Osaka H, Moriyama N, Horita H, Nishiyama K, Yoneda Y, Kondo Y, Tsurusaki Y, Doi H, Miyake N, Hayasaka K, Matsumoto N (2012) CASK aberrations in male patients with Ohtahara syndrome and cerebellar hypoplasia. Epilepsia 53:1441-1449
Scaglia F, Wong LJ, Vladutiu GD, Hunter JV (2005) Predominant cerebellar volume loss as a neuroradiologic feature of pediatric respiratory chain defects. AJNR Am J Neuroradiol 26:1675-80
Schottmann G, Picker-Minh S, Schwarz JM, Gill E, Rodenburg RJT, Stenzel W, Kaindl AM, Schuelke M (2017) Recessive mutation in EXOSC3 associates with mitochondrial dysfunction and pontocerebellar hypoplasia. Mitochondrion 37:46-54
Silver G, Mercimek-Andrews S (2020) Inherited Metabolic Disorders Presenting with Ataxia. Int J Mol Sci 21
Speijer D, Manjeri GR, Szklarczyk R (2014) How to deal with oxygen radicals stemming from mitochondrial fatty acid oxidation. Philos Trans R Soc Lond B Biol Sci 369: 20130446
Srivastava S, McMillan R, Willis J, Clark H, Chavan V, Liang C, Zhang H, Hulver M, Mukherjee K (2016) X-linked intellectual disability gene CASK regulates postnatal brain growth in a non-cell autonomous manner. Acta Neuropathol Commun 4:30
Steinlin M, Blaser S, Boltshauser E (1998) Cerebellar involvement in metabolic disorders: a pattern-recognition approach. Neuroradiology 40:347-54
Stevenson D, Laverty HG, Wenwieser S, Douglas M, Wilson JB (2000) Mapping and expression analysis of the human CASK gene. Mamm Genome 11:934-7
Valayannopoulos V, Michot C, Rodriguez D, Hubert L, Saillour Y, Labrune P, de Laveaucoupet J, Brunelle F, Amiel J, Lyonnet S, Enza-Razavi F, Attie-Bitach T, Lacombe D, Bahi-Buisson N, Desguerre I, Chelly J, Burglen L, Boddaert N, de Lonlay P (2012) Mutations of TSEN and CASK genes are prevalent in pontocerebellar hypoplasias type 2 and 4. Brain 135: e199; author reply e200
van Dijk T, Baas F, Barth PG, Poll-The BT (2018) What's new in pontocerebellar hypoplasia? An update on genes and subtypes. Orphanet J Rare Dis 13
van Dijk T, Barth P, Baas F, Reneman L, Poll-The BT (2021) Postnatal Brain Growth Patterns in Pontocerebellar Hypoplasia. Neuropediatrics 52:163-169
van Dijk T, Rudnik-Schoneborn S, Senderek J, Hajmousa G, Mei H, Dusl M, Aronica E, Barth P, Baas F (2017) Pontocerebellar hypoplasia with spinal muscular atrophy (PCH1): identification of SLC25A46 mutations in the original Dutch PCH1 family. Brain 140: e46
van Dijk T, van Ruissen F, Jaeger B, Rodenburg RJ, Tamminga S, van Maarle M, Baas F, Wolf NI, Poll-The BT (2017) RARS2 Mutations: Is Pontocerebellar Hypoplasia Type 6 a Mitochondrial Encephalopathy? JIMD Rep 33:87-92

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. A method of treating a CASK-linked neurodevelopmental disorder and/or a symptom thereof in a subject in need thereof, the method comprising:
   administering an amount of an SkQ compound or formulation thereof to the subject in need thereof.
2. The method of aspect 1, wherein the SkQ compound is selected from SkQ1, SkQR1, SkQ2, SkQ2M, SkQ3, SkQ4, SkQ5, SkqBerb, SkQPalm. Ct12TPP, MitoQ, and any combination thereof.
3. The method of any one of aspects 1-2, wherein the CASK-linked neurodevelopmental disorder and/or a symptom thereof is a cerebellar hypoplasia.
4. The method of any one of aspects 1-3, wherein the CASK-linked neurodevelopmental disorder and/or a symptom thereof is a Pontocerebellar hypoplasia (PHC).
5. The method of aspect 4, wherein the PHC is a PHC type 1, a PHC type 2, PHC type 3, PHC type 4, PHC type 5, or PHC type 6.
6. The method of aspect 5, wherein the PHC type 1 is PHC type 1A or PHC type 1B.
7. The method of any one of aspects 5-6, wherein the PHC type 2 is PHC type 2A, PHC type 2B, or PHC type 2C.
8. The method of any one of aspects 3-7, wherein the CASK-linked neurodevelopmental disorder and/or a symptom thereof is microcephaly with pontine and cerebellar hypoplasia.
9. The method of any one of aspects 1-8, wherein the CASK-linked neurodevelopmental disorder and/or a symptom thereof is an X-linked intellectual disability.
10. The method of any one of aspects 1-9, wherein the CASK-linked neurodevelopmental disorder and/or a symptom thereof is epilepsy.
11. The method of any one of aspects 1-10, wherein the CASK-linked neurodevelopmental disorder is Rett syndrome.
12. The method of any one of aspects 1-11, wherein administration of the SkQ compound or formulation thereof occurs during a therapeutic temporal window.
13. The method of any one of aspects 1-12, wherein mitochondrial metabolism is improved in the subject in need thereof as measured by a decrease in brain reactive oxygen species in the brain, an increase in brain mitochondrial basal respiration, increase in brain glucose oxidation, a decrease in brain lipid oxidation, an increase in the levels of brain arachidonic acid as compared to the subject in need thereof prior to administration or other suitable control or threshold value.
14. The method of any one of aspects 1-13, wherein neuron loss, neuron dysfunction, neurodegeneration, or any combination thereof is decreased in the subject in need thereof after administration as compared to the subject in need thereof prior to administration or other suitable control or threshold value.
15. A pharmaceutical formulation for treating a CASK-linked neurodevelopmental disorder or a symptom thereof in a subject in need thereof, the pharmaceutical formulation comprising: an amount of an SkQ compound effective to treat a CASK-linked neurodevelopmental disorder or a symptom thereof; and a pharmaceutically acceptable carrier.
16. The pharmaceutical formulation of aspect 15, wherein the SkQ compound is selected from SkQ1, SkQR1, SkQ2, SkQ2M, SkQ3, SkQ4, SkQ5, SkqBerb, SkQPalm. Ct12TPP, MitoQ, and any combination thereof.
17. The pharmaceutical formulation of any one of aspects 15-16, wherein the CASK-linked neurodevelopmental disorder and/or a symptom thereof is a cerebellar hypoplasia.
18. The pharmaceutical formulation of any one of aspects 15-17, wherein the CASK-linked neurodevelopmental disorder and/or a symptom thereof is a Pontocerebellar hypoplasia (PHC).
19. The pharmaceutical formulation of aspect 18, wherein the PHC is a PHC type 1, a PHC type 2, PHC type 3, PHC type 4, PHC type 5, or PHC type 6.
20. The pharmaceutical formulation of 19, wherein the PHC type 1 is PHC type 1A or PHC type 1B.
21. The pharmaceutical formulation of any one of aspects 19-20, wherein the PHC type 2 is PHC type 2A, PHC type 2B, or PHC type 2C.
22. The pharmaceutical formulation of any one of aspects 15-21, wherein the CASK-linked neurodevelopmental disorder and/or a symptom thereof is microcephaly with pontine and cerebellar hypoplasia.
23. The pharmaceutical formulation of any one of aspects 15-22, wherein the CASK-linked neurodevelopmental disorder and/or a symptom thereof is an X-linked intellectual disability.
24. The pharmaceutical formulation of any one of aspects 15-23, wherein the CASK-linked neurodevelopmental disorder and/or a symptom thereof is epilepsy.
25. The pharmaceutical formulation of any one of aspects 15-24, wherein the CASK-linked neurodevelopmental disorder is Rett syndrome.
26. A kit comprising the pharmaceutical formulation of any one of aspects 15-25.

1. A method of treating a CASK-linked neurodevelopmental disorder and/or a symptom thereof in a subject in need thereof, the method comprising:
administering an amount of an SkQ compound or formulation thereof to the subject in need thereof.

2. The method of claim 1, wherein the SkQ compound is selected from SkQ1, SkQR1, SkQ2, SkQ2M, SkQ3, SkQ4, SkQ5, SkqBerb, SkQPalm. Ct12TPP, MitoQ, and any combination thereof.

3. The method of claim 1, wherein the CASK-linked neurodevelopmental disorder and/or a symptom thereof is a cerebellar hypoplasia.

4. The method of claim 1, wherein the CASK-linked neurodevelopmental disorder and/or a symptom thereof is a Pontocerebellar hypoplasia (PHC).

5. The method of claim 4, wherein the PHC is a PHC type 1, a PHC type 2, PHC type 3, PHC type 4, PHC type 5, or PHC type 6.

6. The method of claim 5, wherein (a) the PHC type 1 is PHC type 1A or PHC type 1B; (b) the PHC type 2 is PHC type 2A, PHC type 2B, or PHC type 2C; or (c) both (a) and (b).

7. (canceled)

8. The method of claim 3, wherein the CASK-linked neurodevelopmental disorder and/or a symptom thereof is microcephaly with pontine and cerebellar hypoplasia.

9. The method of claim 1, wherein the CASK-linked neurodevelopmental disorder and/or a symptom thereof is an X-linked intellectual disability.

10. The method of claim 1, wherein the CASK-linked neurodevelopmental disorder and/or a symptom thereof is epilepsy.

11. The method of claim 1, wherein the CASK-linked neurodevelopmental disorder is Rett syndrome.

12. The method of claim 1, wherein administration of the SkQ compound or formulation thereof occurs during a therapeutic temporal window.

13. The method of claim 1, wherein mitochondrial metabolism is improved in the subject in need thereof as measured by a decrease in brain reactive oxygen species in the brain, an increase in brain mitochondrial basal respiration, increase in brain glucose oxidation, a decrease in brain lipid oxidation, an increase in the levels of brain arachidonic acid as compared to the subject in need thereof prior to administration or other suitable control or threshold value.

14. The method of claim 1, wherein neuron loss, neuron dysfunction, neurodegeneration, or any combination thereof is decreased in the subject in need thereof after administration as compared to the subject in need thereof prior to administration or other suitable control or threshold value.

15. A pharmaceutical formulation for treating a CASK-linked neurodevelopmental disorder or a symptom thereof in a subject in need thereof, the pharmaceutical formulation comprising:
an amount of an SkQ compound effective to treat a CASK-linked neurodevelopmental disorder or a symptom thereof; and
a pharmaceutically acceptable carrier.

16. The pharmaceutical formulation of claim 15, wherein the SkQ compound is selected from SkQ1, SkQR1, SkQ2, SkQ2M, SkQ3, SkQ4, SkQ5, SkqBerb, SkQPalm. Ct12TPP, MitoQ, and any combination thereof.

17. The pharmaceutical formulation of claim 15, wherein the CASK-linked neurodevelopmental disorder and/or a symptom thereof is a cerebellar hypoplasia.

18. The pharmaceutical formulation of claim 15, wherein the CASK-linked neurodevelopmental disorder and/or a symptom thereof is a Pontocerebellar hypoplasia (PHC), optionally wherein the PHC is a PHC type 1, a PHC type 2, PHC type 3, PHC type 4, PHC type 5, or PHC type 6.

19. (canceled)

20. The pharmaceutical formulation of claim 19, wherein (a) the PHC type 1 is PHC type 1A or PHC type 1B (b) the PHC type 2 is PHC type 2A, PHC type 2B, or PHC type 2C; or (c) both (a) and (b).

21. (canceled)

22. The pharmaceutical formulation of claim 15, wherein (a) the CASK-linked neurodevelopmental disorder and/or a symptom thereof is microcephaly with pontine and cerebellar hypoplasia; (b) the CASK-linked neurodevelopmental disorder and/or a symptom thereof is an X-linked intellectual disability; (c) the CASK-linked neurodevelopmental disorder and/or a symptom thereof is epilepsy: (d) the CASK-linked neurodevelopmental disorder is Rett syndrome; or (e) any combination of (a)-(d).

23. (canceled)

24. (canceled)

25. (canceled)

26. A kit comprising the pharmaceutical formulation of claim 15.
